(19) United States
(12) Patent Application Publication
(10) Pub. No.: US 2010/0024643 A1
(43) Pub. Date: Feb. 4, 2010

(54) COATING PROCESS AND COATED PRODUCT

(76) Inventor: Robert Harold Bradley, Aberdeen (GB)

Correspondence Address: TAFT, STETTINIUS & HOLLISTER LLP, SUITE 1800, 425 WALNUT STREET, CINCINNATI, OH, 45202-3957, US

(21) Appl. No.: 12/373,273

(22) PCT Filed: Jul. 16, 2007
(86) PCT No.: PCT/GB2007/002679

§ 371 Date: Jun. 15, 2009

(30) Foreign Application Priority Data
Jul. 17, 2006 (GB) 0614081.8
Jul. 21, 2006 (GB) 0614475.2

(51) Int. Cl. B01D 53/04; B01J 20/26; A61M 16/10

(52) U.S. Cl. 95/139; 96/154; 502/402; 128/205.27

(57) ABSTRACT

A process modifies the surface diffusion and wetting characteristics of a microporous material such as active carbon or silica, by applying a plasma of polymer precursor monomers to an external surface of the material to modify its adsorption properties, the microporous material obtainable thereby being modified by presence of a nanolayer of polymeric material extending over said external surface around the pores to partially occlude pore openings to a predetermined extent, but the nanolayer does not substantially infiltrate the pores of the microporous material.

### TECHNICAL FIELD

The present invention lies in the field of fluid adsorption and separation, especially concerning novel materials for separating gases and volatile materials.

In particular the invention relates to a process for modifying the properties of a microporous material, in particular a process for modifying the surface diffusion and wetting characteristics of a microporous material in a controllable and predictable manner. There is also described a microporous material comprising a nanolayer of material wherein the nanolayer does not substantially infiltrate the pores of the microporous material.

### BACKGROUND ART

Activated carbon is associated with excellent properties for a wide range of adsorption and separation applications. Microporous material formed from activated carbon is characterised by high adsorptive capacity, wherein most of the adsorptive capacity occurs in micropores (typically having a mean diameter of less than 2 nm) and such microporous material is also associated with a strong affinity for organic compounds. The surface of microporous material formed from activated carbon is generally essentially non-polar, and these materials are thus hydrophobic and organophilic. However, the adsorption properties associated with such microporous material are strongly influenced by the presence of large amounts of adsorbed oxygen, which increases the hydrophilicity of the surfaces. In practical applications this frequently means that the adsorption of an adsorbate such as an organic compound by the microporous material is compromised because of the co-adsorption of water vapour.

The surface of activated carbon is essentially non-polar, making it hydrophobic and organophilic. However, the adsorption properties of such material are strongly influenced by the presence of large amounts of absorbed water. The apparent anomaly arises because the concentration of water vapour is generally much greater than that of any gaseous or vaporous contaminants in the air.

An object of the present invention is to provide an improved adsorbent, and a process for producing same whereby at least some of the aforesaid problems are obviated or mitigated.

This object is achievable by the invention to be described hereinbelow, by modification of the surface of the adsorbent material.

### SUMMARY OF THE INVENTION

Thus according to a first aspect of the present invention there is provided a process of modifying the properties of a microporous material comprising the steps of:
converting a composition comprising a monomer to the form of a plasma;
initiating polymerisation of the composition; and
applying a nanolayer of the plasma composition to a surface of the microporous material to form a modified microporous material.

Generally, the properties of a surface of the microporous material are modified through A process according to the present invention; in particular the chemical and physical properties of the surface of the microporous material. Surprisingly the properties of the interior of the microporous material are not affected by the method of the present invention, and the bulk properties of the modified microporous material are suitably identical to the properties of the microporous material prior to modification. In particular, the bulk chemical and physical properties of the microporous material are suitably identical to the microporous material before modification.

Adoption of this process provides a surface modification wherein the processed microporous material has external surfaces to which the nanolayer is applied, but the internal surfaces within the microporous structure are substantially free of such surface modification. Furthermore, the applied nanolayer extends over said external surface around the pores to partially occlude same to a predetermined extent.

Accordingly the present invention provides a process for modifying the surface properties of a microporous material whilst maintaining the bulk properties of the microporous material.

Typically, the surface diffusion and wetting characteristics of the surface of the microporous material are modified through A process according to the present invention.

Generally the adsorption properties of the microporous material are modified. Suitably the adsorption properties are controllably and predictably adjusted.

Typically A process according to the present invention increases the hydrophobicity of the surface of the microporous material. Typically the hydrophobicity of the surface is doubled. The hydrophobicity of the microporous material may be investigated using immersion calorimetry, suitably with water as the probe. It is generally acknowledged that heats of immersion in water provide an indication of hydrophobicity, where an increase in heat of immersion in water indicates an increase in hydrophobicity. Typically A process according to the present invention decreases the heat of immersion in water associated with the surface from −40 mJm⁻² to −15 mJm⁻², suitably the heat of immersion in water associated with the surface is decreased to −10 mJm⁻².

Typically, the surface adsorptive properties of the microporous material are altered. Normally, the processed material offers selective entry to the pores of the microporous material depending on the kinetic energy and/or the molecular size of the molecules to be adsorbed (the adsorbate), where the greater the kinetic energy of the adsorbate molecule and/or the smaller the size of the adsorbate molecule, the more easily it will be adsorbed by the modified microporous material. Thus appropriate control of the coating process allows the microporous material to be designed to selective with respect to an intended adsorbate. Entry to the pores of the microporous material may be selectively controlled by the material having regard to the polarity of the adsorbate where the less polar the adsorbate molecule is, the more easily it will be adsorbed into the modified microporous material.

An advantage offered by the process lies in the fact that the nanolayer is applied to external surfaces, and surrounding the lip of the pores therein to a limited extent, so that the interior surfaces of the microporous material are substantially free of such nanolayer. This has the natural consequence that the capacity of the pores of the modified microporous material is not greatly affected by A process according to the present invention, and generally the capacity of the modified microporous material is at least 90% of the capacity of the unmodified microporous material.

Therefore, application of the process provides that the nanolayer remains on the surface of the microporous material and does not penetrate into the pores of the microporous material to any significant extent.

The nanolayer of surface modifying material is applied such that polymerisation is preferably initiated during conversion of the precursor composition to a plasma. Alternatively polymerisation may be initiated before the precursor composition is converted into a plasma.

Suitably, the average pore diameter of the microporous material is 2 nm or less; generally less than 1.5 nm. Typically more than 50% of the pores of the microporous material have a diameter of less than 2 nm; suitably more than 80% have a diameter of less than 2 nm.

The process offers control over the modification of the surface properties of the material by selection of appropriate nanolayer precursors, and control of the polymerization and plasma deposition steps. Thus for example an adjustment in terms of hydrophobic or hydrophilic characteristics of the external surfaces is achievable by considering the chemical properties of the available precursor materials, and thus is controllable by selecting precursors containing elements offering the appropriate chemical properties (Si, F, O etc.).

Similarly the extent of occlusion of the pores, or pore entry ‘gate’ effect , is a mainly a physical property of the deposited nanolayer and is controllable by the deposition process directly so as to leave a pre-determined gap for entry into the pores. The restriction access to the pores by controlled coating of the exterior surface lip around the pore is mainly controlled by he plasma deposition rate and how much constriction is induced, and that is a function of precursor in-flow, plasma power, and the overall treatment time, which may be of the order of about an hour or less. That is not to say that pore access does not involve secondary chemistry-based repulsion/attraction effects dependant upon the nature of the species entering the microporous material and the composition of the deposited layer.

The material is usefully provided as particulate material, such as granular or fibre forms wherein the size may range from nm to several mm for the maximum dimension of each particle. In fibre form the diameter of the fibre may be of the order of about 7 microns (μm).

The processed material may be adopted for use after formation of the nanolayer, or may be post-processed in a further thermal stabilisation step to improve properties such as refractory characteristics.

Typically the process is applicable to microporous material such as silicon, carbon or activated silicon or activated carbon. Advantageously the microporous material is activated carbon as such material is associated with excellent properties for a wide range of adsorption and separation applications. Suitably the microporous material may be a bituminous lignite-based carbon.

The process typically produces a material having a composition that is typically hydrophobic.

The process may employ a step of introducing a precursor monomer comprising an element intended to provide a surface modifying effect. Thus the composition of precursor monomers to be polymerised may include any monomer comprising silicon, or oxygen, or a halogen such as chlorine, or fluorine, or a pendant group conferring a desired surface-modifying property.

Useful organic precursors for inclusion in the polymerisable composition include for example: hexadimethylsiloxane, other silanes and Si containing organic compounds, chloro- and fluorohydrocarbons such as fluorohexane or other F-containing organics and other CFCs/Freon®-type molecules. In particular the precursor composition may comprise hexamethyldisiloxane (HMDSO) or perfluorohexane (PFH). Advantageously the composition comprises HMDSO. HMDSO polymer has an associated resistance to water permeation similar to that associated with polysiloxane films.

When used herein the term “plasma” is intended to mean an ionised gas consisting of free electrons ions and neutral atoms. Plasmas are generally formed when sufficient energy is applied to a gas. The technique of forming a plasma is generally known and the application thereof in forming specialist coatings is referred to as plasma-enhanced chemical vapour deposition (PECVP).

Suitably the method of transforming the composition into a plasma involves providing the composition in the form of a vapour and applying a sufficient electric potential across the vapour to transform the composition into a plasma.

The plasma formation method may be a capacitive coupling or an inductive coupling method; power is typically coupled into the vaporised composition inductively or capacitively.

Typically the plasma deposition method occurs at constant power, suitably a power of 20 to 60 W; more suitably at a power of 40 W or more.

Furthermore, the method of transforming the composition into a plasma typically takes place in a closed chamber. Suitably the vaporised composition is introduced into the chamber at a constant flow rate.

Normally, the method of transforming the composition into a plasma takes place under a Vacuum, typically a vacuum of 0.6 nmHg.

Generally during plasma formation the pressure under which the reaction takes place is varied.

A magnetic field may be applied during plasma formation. The application of a magnetic field means that the strength of the electric potential applied across the vaporised composition may be decreased without a decrease in the rate of conversion to a plasma.

A gas may be introduced during plasma formation, the gas is generally an inert gas. Alternatively the gas may be a reactive gas, wherein the reactive gas may react with the composition, typically to introduce functional groups thereto.

A process according to plasma formation may include the addition of an oxidant. Any known oxidant may be introduced.

Suitably the composition is applied to the microporous material through a plasma enhanced chemical vapour deposition method.

The composition in plasma form is generally applied to a surface of the microporous material for 15 minutes or less; suitably 10 minutes or less; more suitably 1, 5 or 10 minutes. Typically the composition in plasma form is applied to the surface of the microporous material for 1 to 2 minutes.

The composition in plasma form is suitably applied to the microporous material until a layer of composition having a thickness of 1 to 50 nm is present on the surface of the microporous material.

The composition is suitably applied to the microporous material at a flow rate of 40 to 80 standard cubic centimetres per minute (Sccm), advantageously 60 Sccm.

A process according to this invention thus provides a new microporous material with modified surface properties.

Thus according to a second aspect of the invention there is provided a microporous adsorbent particulate material having internal and external surfaces, said external surfaces

(i) having pores therein capable of admitting fluids, and

(ii) being modified by presence of a nanolayer of a polymeric material; said nanolayer extending over said external surface around a pore to partially occlude same to a predetermined extent;

said internal surfaces being substantially free of said polymeric material.

The nanolayer is suitably formed from a polymerisable composition comprising at least one precursor monomer comprising an element intended to provide a surface modifying effect. Thus the composition of precursor monomers to be polymerised may include any monomer comprising silicon, or oxygen, or a halogen such as chlorine, or fluorine, or a pendant group conferring a desired surface-modifying property.

Useful organic precursors for inclusion in the polymerisable composition include for example: hexadimethylsiloxane, other silanes and Si containing organic compounds, chloro- and fluorohydrocarbons such as fluorohexane or other F-containing organics and other CFCs/Freon®-type molecules. In particular the precursor composition may comprise hexamethyldisiloxane (HMDSO) or perfluorohexane (PFH). Advantageously the composition comprises HMDSO. HMDSO polymer has an associated resistance to water permeation similar to that associated with polysiloxane films.

The bulk microporous adsorbent material to form the basis for such a nanolayer-coated particulate material may be silicon, carbon or activated silicon or activated carbon.

The microporous adsorbent material of this invention is characterised by surface modifications which alter the external properties of the material but leave the internal bulk properties substantially unchanged.

Particularly, the material is rendered selective with regard to adsorption characteristics by a combination of chemical modifications and physical barrier attributes arising from partial occlusion of the external surface openings of the pores in the microporous material.

Thus in one embodiment, the microporous adsorbent material comprises a particulate material that is a carbon-based microporous material, and said internal surfaces exhibit properties associated with microporous carbon.

In another embodiment, the microporous adsorbent material comprises a particulate material that is a silicon-based microporous material, and said internal surfaces exhibit properties associated with microporous silicon.

The microporous adsorbent material may comprise a nanolayer polymeric external coating wherein the polymeric material is a silicon-based material and said external surfaces exhibit properties associated with silicon.

In another embodiment, the microporous adsorbent material comprises a particulate material wherein the bulk of the microporous material consists of carbon or silicon, and the surface nanolayer comprises at least one element conferring enhanced hydrophobic properties to said surface nanolayer.

In yet another embodiment, the microporous adsorbent material comprises a particulate material wherein the bulk of the microporous material consists of carbon or silicon, and the surface nanolayer comprises at least one element conferring enhanced hydrophilic properties to said surface nanolayer.

A microporous adsorbent material of this invention may have a surface nanolayer that comprises a halogen, such as chlorine or fluorine.

The microporous adsorbent material of this invention may have a nanolayer that comprises a surface modifying compound to enhance hydrophobic properties of the external surfaces of the material.

Such a compound may be a halocarbon, preferably a fluorocarbon such as perfluorohexane.

The form of the microporous adsorbent material may be a granular particulate form, including a powder form, or it may be in fibre form.

The microporous adsorbent material may be such that the largest dimension of the particulate material has a size range of the order of nm to several mm.

The microporous adsorbent material may have a nanolayer that has thickness in the range of from 1 to 1000 nm.

The microporous adsorbent material may be one where the nanolayer comprises a hydrophobic polymer.

Alternatively, the microporous adsorbent material may be one wherein the nanolayer comprises a hydrophilic polymer

Preferably, the microporous adsorbent material is one in which the nanolayer is a plasma enhanced chemical vapour deposit.

The nanolayer may be selectively altered by appropriate use of suitable precursor materials to confer enhanced surface properties, particularly to adjust hydrophobic and hydrophilic properties.

The microporous adsorbent material may be one in which the nanolayer is a polymer derived from polymerisable organic precursors such as hexadimethylsiloxane, other silanes and Si containing organics, halohydrocarbons, fluorohexane and other F-containing organics and other CFCs/Freon type molecules or one in which the nanolayer is a polymer derived from oxygen-functionalised organics.

The microporous adsorbent material may be used as such for loose-fill packing of a container, or suitably attached to a support which may be a conformable to a desired shape, or may be a rigid support, e.g. a tubular component.

When a support is used, it may be in the form of fibres, non-woven fibre cloths, woven fibre cloths, flexible films and the like.

In an embodiment a support may be a fluid-permeable body.

A suitable support may be a carbon monolith.

The microporous adsorbent material may be incorporated in to a filter device as a loose fill or upon a support element.

Further according to the invention there is provided a bulk storage device for fluids comprising a container filled with microporous adsorbent material in accordance with the second aspect of the invention.

Still further according to the invention there is provided a separation system for selectively extracting one or more fluids comprising a filter device incorporating the microporous adsorbent material in accordance with the second aspect of the invention and a bulk storage device for fluids comprising a container filled with microporous adsorbent material in accordance with the second aspect of the invention, operatively connected such that selectively extracted fluid from the filter device is transferable to said bulk storage device.

In such a separation system the filter device preferably comprises a microporous material that is adapted to selectively adsorb a gas, which may be methane, or in other embodiments the gas may be carbon dioxide, and in others the gas is hydrogen.

In one embodiment the modified microporous material allows selective adsorption of an adsorbate dependent on the kinetic energy, molecular size and/or polarity of the adsorbate.

Suitably the polymer layer penetrates into the pores of the microporous material less than 10% of the depth of the pores; typically less than 1% of the depth of the pores. Generally the polymer layer penetrates into the pores of the microporous material less than 1 nm; typically less than 0.5 nm; more suitably less than 0.1 nm.

Suitably the polymer layer is present on at least one surface of the microporous material and the polymer layer penetrates less than 90% of the pores on the surface of the microporous material; typically less than 95% of the pores; generally less than 99% of the pores.

Generally the polymer layer does not completely cover the pores on the surface of the microporous material, and the pores remain partially or fully open. Typically at least 90% of the apertures of the surface of the microporous material are not closed by being covered with the polymer layer, more typically at least 95% of the apertures.

Typically the entrances of the pores of the modified microporous material are modified compared to unmodified microporous material. The entrances of the pores of the modified microporous material are suitably constructed and narrowed relative to unmodified microporous material.

Typically the entrances to the pores of the microporous material of the present invention are narrowed by 10%; suitably 20%; more suitably by 50%. Advantageously the entrances to the pores of the microporous material of the present invention are narrowed by 70% or more.

Typically the entrances of at least 50% of the pores of the microporous material of the present invention are constricted; suitably at least 70% of the pores are constricted. Typically the polymer layer extends over the edges of the pores on the surface of the microporous material, thus constricting the entrance to the pores.

According to a further aspect of the present invention there is provided a carbonisation method for forming wholly carbon-containing adsorbents using the microporous material as described above as a precursor.

Suitably the wholly carbon-containing adsorbents have similar properties to those associated with the abovementioned microporous material and much higher thermal stability than that associated with the abovementioned microporous material.

Typically the microporous material for use as a precursor is PECVD polymer treated carbon.

According to a further aspect of the present invention there is provided a modified microporous material as described above having adsorbate material adsorbed therein.

The constructions to the entrances of the pores of the modified microporous material typically act as kinetic energy “gates” wherein the adsorbate molecules must have sufficient energy to overcome the constrictions to the entrances of the pores in order to enter the pores. Pore entry is thus dependent on the kinetic energy of the adsorbate molecules. Suitably increasing the temperature of adsorption increases the amount of adsorbate adsorbed.

According to one embodiment entry to the pores of the surface of the modified microporous material is dependent on the molecular size of the adsorbate molecule. Increasing the molecular size of the adsorbate molecule decreases the amount of adsorbate molecule which enters the pores of the microporous material, thus decreasing the amount of adsorbate molecule adsorbed.

Suitably entry to the pores of the surface of the modified microporous material is dependent on the polarity of the assorbate where increasing the polarity of the absorbate decreases the amount of adsorbate adsorbed.

Diffusion and adsorption into the microporous material is suitably selective and controllable. Suitably diffusion and adsorption may be controlled by controlling the kinetic energy of the adsorbate molecule and/or altering the molecular size of the adsorbate. Alternatively, diffusion and adsorption may be controlled by altering the polarity of the adsorbate.

A dramatic decrease of the amount of adsorbate material adsorbed by the microporous material may be observed by decreasing the kinetic energy of the adsorbate material, typically by reducing the temperature of the adsorbate material. The amount of adsorbate material adsorbed by the microporous material may also be decreased by increasing the molecular size of the adsorbate material.

Typically the capacity of the pores of the microporous material of the present invention does not differ greatly from unmodified microporous material. Suitably the capacity of the modified microporous material is at least 90% of the capacity of the non-modified material; more suitably at least 95% of the capacity of the non-modified material.

Small loses of adsorption capacity in terms of total and micropore volumes may be explained in terms of molecular packing effects within the pore structure possibly coupled with some molecular shifting.

Typically the adsorbate is nitrogen, an organic molecule such as an alkyl molecule or an alcohol. The adsorbate is suitably nitrogen, C₇H₈, methanol, ethanol or propanel. Alternatively the adsorbate may be nerve gas.

Suitably the adsorbate molecules have an average molecular diameter of 1 to 2 nm; more suitably an average molecular diameter 1 to 1.5 nm.

Suitably the absorbate molecule is nitrogen and is at a temperature of more than 100K.

Alternatively the adsorbate molecule is an organic compound such as C₇H₈ methanol, ethanol or propanol, at a temperature of 250K or more, suitably 300K or more.

The adsorption data associated with microporous materials may be analysed using the Dubinin-Radushkevish equation:

W=W₀exp[−(A/βE₀)²]

Where W is the volume of liquid like adsorbate within the pore structure at relative p/p^{s} and W₀ is the micropore volume. A=RTlnP⁰/P is the thermodynamic adsorption potential required to bring 1 mole of adsorbate to the state of the bulk liquid at absolute temperature. R is the gas content and E₀ the characteristic adsorption energy which is a function of the adsorbate. β is the so called affinity coefficient which depends on the adsorptive. By convention β(C₆H₆)=1. On the basis of molecular sieve experiments it has been shown that for active carbon the characteristic energy E₀(KJ mol⁻¹) is related to the average width L of the slit shaped micropores (Stoeckli 1995).

L(nm)=10.8/E₀−11.4)

For slit shaped micropores as found in active carbons, the surface area of the pores is related to their volume and their width through:

Sₘᵢ(m²·g⁻¹)=2.10³−W₀(cm³·g⁻¹)/L(nm)

The capacity of the microporous material may be investigated using standard liquid density data.

According to a further aspect of the present invention there is provided the use of the modified microporous material as described above in a method of selectively adsorbing a molecule, particularly in a humid environment.

In particular, the modified microporous material may be used in a method of recovering hydrogen from air, removing toxic gases such as nerve gas from air, gas storage and other specialist separations in the pharmaceutical and biomedical fields.

According to a further aspect of the present invention there is provided the use of the modified microporous material as described above in the manufacture of medical apparatus, such as breathing apparatus.

According to a further aspect of the present invention there is provided breathing apparatus comprising the modified microporous material as described above.

Suitably the breathing apparatus removes gases toxic to humans or animals from air, typically nerve gas.

### BRIEF DESCRIPTION OF DRAWINGS

The present invention will now be described by way of example only with reference to the accompanying figures in which:

FIG. 1a shows an adsorption isotherm of nitrogen at 77K for activated carbon (BPL), and three modified BPL microporous materials prepared according to Example 1;

FIG. 1b shows a Dubinin-Radushekevich plot relating to the adsorption of nitrogen at 77K for activated carbon (BPL), and three modified BPL microporous materials prepared according to Example 1;

FIG. 2a shown an adsorption isotherm of methanol at 303K for activated carbon (BPL), and three modified BPL microporous materials prepared according to Example 1;

FIG. 2b shows a Dubinin-Radushekevich plot relating to the adsorption of methanol at 303K for activated carbon (BPL), and three modified BPL microporous materials prepared according to Example 1;

FIG. 3a shows an adsorption isotherm of ethanol at 303K for activated carbon (BPL), and three modified BPL microporous materials prepared according to Example 1;

FIG. 3b shows a Dubinin-Radushekevich plot relating to the adsorption of ethanol at 303K for activated carbon (BPL), and three modified BPL microporous materials prepared according to Example 1;

FIG. 4a shows an adsorption isotherm of isopropanol at 303K for activated carbon (BPL), and three modified BPL microporous materials prepared according to Example 1;

FIG. 4b shows a Dubinin-Radushekevich plot relating to the adsorption of isopropanol at 303K for activated carbon (BPL), and three modified BPL microporous materials prepared according to Example 1;

FIG. 5a shows an adsorption isotherm of toluene at 30K for activated carbon (BPL), and three modified BPL microporous materials prepared according to Example 1;

FIG. 5b shows a Dubinin-Radushekevich plot relating to the adsorption of toluene at 303K for activated carbon (BPL), and three modified BPL microporous materials prepared according to Example 1.

### DESCRIPTION OF MODES FOR CARRYING OUT THE INVENTION

### EXAMPLE 1

1 g of microporous activated carbon, BL a bituminous Pittsburgh ligite based material was exposed to HMDSO (Sigma, Aldrich) Plasma and PFH plasma in a plasma chamber. An RF power of 40 W and a constant flow rate of circa 60 Sccm for HMDSO was used in all experiments for deposition times of 1, 5 and 10 minutes respectively.

The surface chemical composition of HMDSO plasma treated BPL was studied using a Kratos Axix His 5 channel imaging X-ray photoelectron spectrometer using monochromated Alkα radiation (1486.6.ev). A Calorimeter calvet 80 C was used to measure the heat of immersion. Measurements were performed at room temperature (25+2° C.) using three test liquids, distilled water (laboratory prepared), methanol and isopropanol. The parameter characteristics of unmodified BPL (BPL-0) and BPL plasma treated as described above at deposition times of 1, 5 and 10 minutes respectively (BPL-1, BPL-2 and BPL-3) were determined in an automated volumetric gas adsorption apparatus (ASAP 2001). 0.3 g of BPL-0, BPL-1, BPL-2 and BPL-3 were outgassed for 22 hours at 353K. The adsorption temperature was maintained using liquid N₂ (77K).

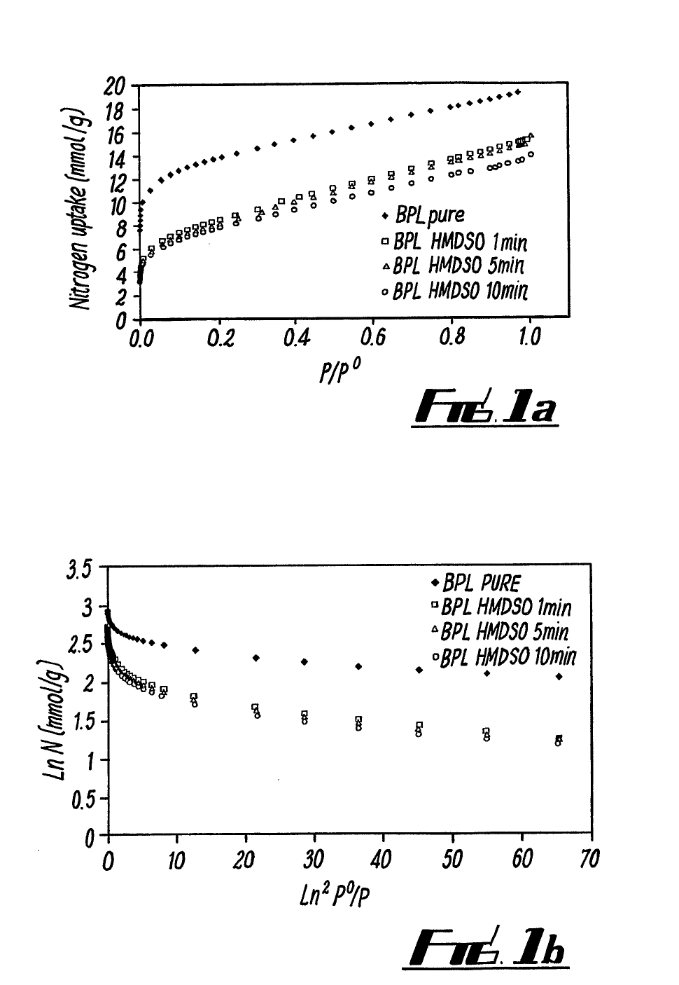

The adsorption isotherm of N₂ at 77K on BPL-1, BPL-2 AND BPL-3 is presented in FIG. 1a. All nitrogen isotherms have a shape belonging to type I of the IUPAC classification as shown in FIG. 1a.

All BPL modified by HMDSO plasma (BPL-1, BPL-2 and BPL-3) maintain the characteristics of a micropore active carbon as shown in FIG. 1a. All the isotherms have been analysed using the Dubinin-Radushekevich approach and FIG. 1b shows the data for the BPL-1, BPL-2 and BPL-3 plotted in the form of Eq. (1).

FIG. 1b shows an upward deviation apparent at high values of relative pressure, or at low values of LnN²P⁰/P≈20, type C behaviour.

The intercept of this linear portion of the plot on the ordinate LnN²P⁰/P≈0 provides an estimate of micropore volume, because at that pressure all micropores should be filled. As shown in FIG. 1b the decrease in this intercept of the linear part of the D-R plot for BPL-1, BPL-2 and BPL-3 shows a reduction of microporosity. The micropore volume (W₀) of BPL-1, BPL-2 and BPL-3 is up to 40% smaller than BPL-0. This can be interpreted as evidence of the so called molecular sieving effect attributable to a restriction of the entrance to the micropore due to the nanolayer polymer deposited. Nitrogen molecules do not have sufficient kinetic energy to enter micropores smaller than 1.64 nm as presented in Table 1. BPL-1, BPL-2 and BPL-3 exhibit a decrease in surface area of micropores compared to BPL-0 that was calculated using the BET method in a region relative pressure of (0.01-0.2) as shown in Table 1.

Also characteristic adsorption Energy E₀ decrease for BPL-1, BPL-2 and BPL-3 may indicate a less homogeneous micropore structure compared to that of BPL-0 and therefore a higher average pore width as calculated using eqn 2 (see above).

The total pore volumes were obtained from N₂ adsorption isotherms at relative pressure of 0.995 after the conversion of adsorbed amounts to liquid volumes and as shown in Table 1. BPL-1, BPL-2 and BPL-3 show a slight decrease in adsorption capacity as compared to BPL-0. This decrease in adsorption capacity as compared to BPL-0. This decrease in adsorption capacity can be attributable to the decrease in micropore surface area as above explained.

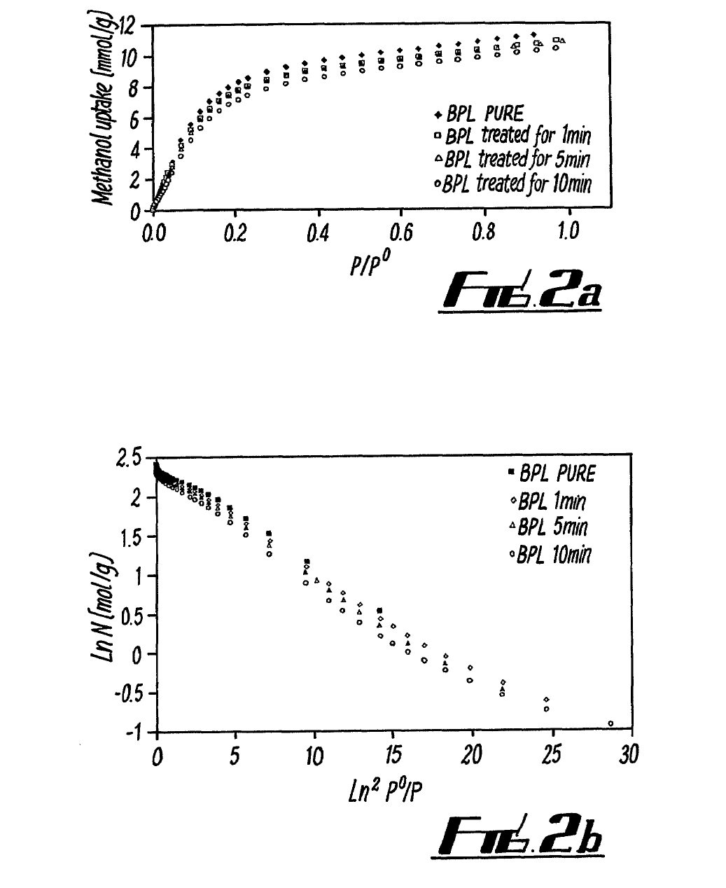

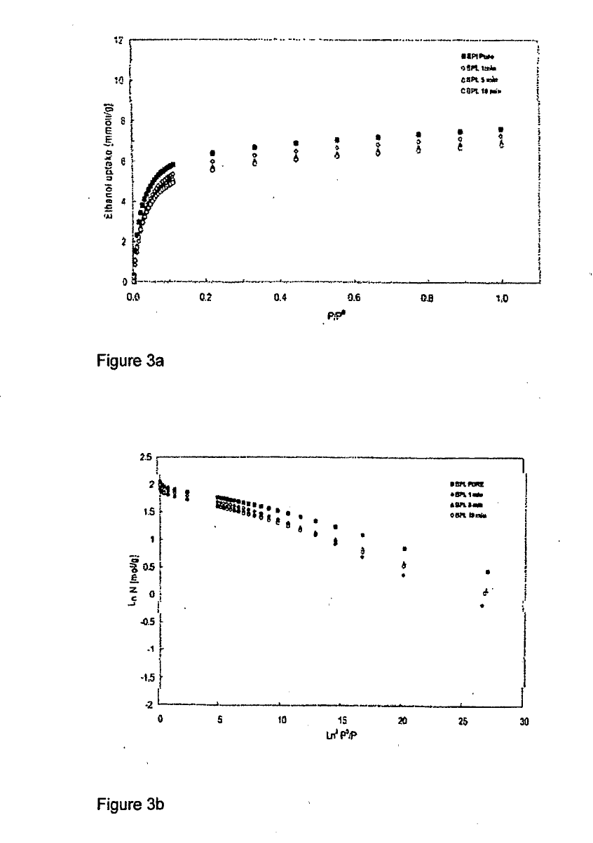

As for the nitrogen isotherm the isotherm for alcohols are overall type I as shown in FIGS. 2a, 3a and 4a.

The plots for adsorption for each alcohol show the same features as those already discussed for nitrogen. For methanol adsorption, increasing the deposition time results in a slight decrease of total pore volume as shown in Table 2. FIG. 2b shows an upward deviation apparent at high values of relative pressure, or at low values of LnN²P⁰/P≈0.26, type C behaviour, and apparent negative deviation for value in LnN²P⁰/P≧5, type D behaviour. Extrapolation of the DR line in the range of 0.26≦LnN²P₀/P≧5 allow the calculation of micropore volume W₀ that slightly decreases with deposition time. Characteristic adsorption energy also decreases. This indicates a minor decrease in adsorption capacity of BPL modified by HMDSO plasma, probably due to so called molecular sieving effect as shown in the case of nitrogen adsorption but in this case the effect of coating onto micropore surface does not induce a big change in micropore volume regardless of the fact that the slit width is slightly bigger than BPL-0 as presented in Table 2.

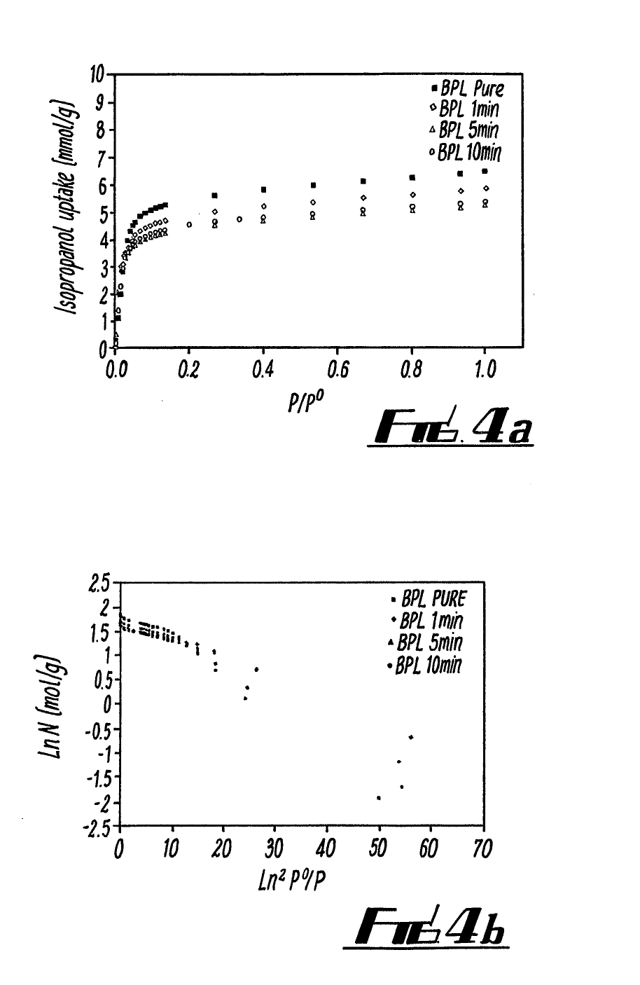

The same analysis and considerations can be conducted relating to ethanol adsorption. Table 3 shows that total pore volume Vₚ, and micropore volume W₀ don't change significantly for BPL modified by HMDSO plasma compared with BPL-0. As for methanol and ethanol adsorption, isopropanol adsorption exhibits an increase in characteristic adsorption energy with increasing deposition time. This may be due to the fact that the coating exhibits hydrophobic behaviour and that isopropanol is less than methanol. This allows isopropanol to enter pores with an entrance of 1.40 nm despite the fact that the total pore volume and the micropore volume seem to decrease with deposition time as presented in Table 4. The relevant DR plot shows that in the region of low relative pressure and exactly for a value in LnN²P⁰/P≈0.26 the plot of the plot for p1 BPL-0 (see FIG. 4b).

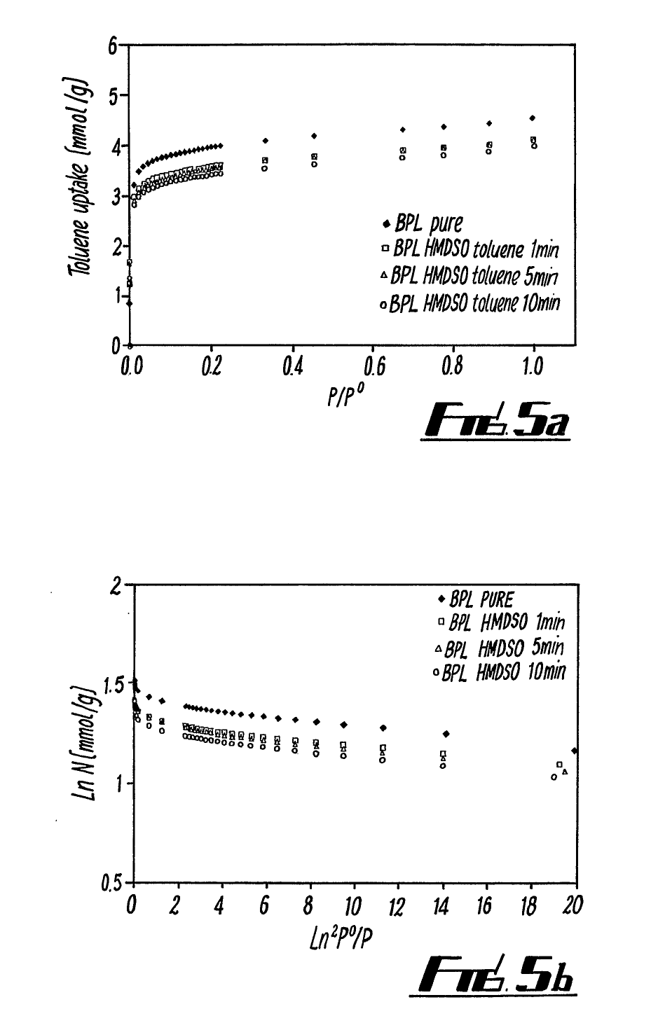

Additionally, all toluene isotherms have an associated shape belonging to type I of the iupac classification as shown in FIG. 5a. FIG. 5b shows an upward deviation apparent at high values of relative pressure, or at low values of LnN²P⁰/P≈4, type C behaviour. The fact that toluene is a non-polar molecule, essentially immiscible in water may explain the high values of adsorption energy associated with toluene as shown in Table 5.

TABLE 1
Table 1: Comparison of characteristic parameters from
eq[1] for BPL-0 and BPL-1, BPL-2 and BPL-3 modified with
HMDSO plasma polymer for 3 different deposition times (1,
5, 10 min respectively) from nitrogen adsorption at 77 K.

| | | | | | | | BET |
|---|---|---|---|---|---|---|---|
| | Plasma | V | W₀ | E₀ | | | Surface |
| | Deposition | [cm³/ | [cm³/ | [KJ/ | L | Sₘᵢ | Area |
| Sample | Time (min) | g] | g] | mol] | [nm] | [m²/g] | [m²/g} |
| BPL | — | 0.46 | 0.40 | 20.47 | 1.19 | 674 | 1049 |
| BPL | 1 | 0.41 | 0.22 | 16.84 | 1.98 | 284 | 672 |
| HMDSO | | | | | | | |
| BPL | 5 | 0.42 | 0.21 | 16.84 | 1.98 | 215 | 659 |
| HMDSO | | | | | | | |
| BPL | 10 | 0.37 | 0.19 | 17.95 | 1.64 | 235 | 618 |
| HMDSO | | | | | | | |

TABLE 2
Table 2: Comparison of characteristic parameters from
eq[1] (see above) for BPL-0 and BPL-1, BPL-2 and BPL-3
modified with HMDSO plasma polymer for 3 different
deposition times (1, 5, 10 min respectively) from
methanol adsorption at 303 K.

| | Plasma | | | | | |
|---|---|---|---|---|---|---|
| | Deposition | V | W₀ | E₀ | L | Sₘᵢ |
| Sample | Time (min) | [cm³/g] | [cm³/g] | [KJ/mol] | [nm] | [m²/g] |
| BPL | — | 0.45 | 0.42 | 18.47 | 1.52 | 548 |
| BPL | 1 | 0.44 | 0.41 | 16.87 | 1.97 | 413 |
| HMDSO | | | | | | |
| BPL | 5 | 0.44 | 0.41 | 16.87 | 1.97 | 413 |
| HMDSO | | | | | | |
| BPL | 10 | 0.42 | 0.39 | 16.42 | 2.14 | 362 |
| HMDSO | | | | | | |

TABLE 3
Table 3: Comparison of characteristic parameters from
eq[1] (see above) for BPL-0 and BPL-1, BPL-2 and BPL-3
modified with HMDSO plasma polymer for 3 different
deposition time (1, 5, 10 min respectively) from ethanol
adsorption at 303 K.

| | Plasma | | | | | |
|---|---|---|---|---|---|---|
| | Deposition | V | W₀ | E₀ | L | Sₘᵢ |
| Sample | Time (min) | [cm³/g] | [cm³/g] | [KJ/mol] | [nm] | [m²/g] |
| BPL | — | 0.44 | 0.41 | 18.78 | 1.46 | 560 |
| BPL | 1 | 0.42 | 0.39 | 17.50 | 1.76 | 441 |
| HMDSO | | | | | | |
| BPL | 5 | 0.41 | 0.37 | 17.26 | 1.84 | 409 |
| HMDSO | | | | | | |
| BPL | 10 | 0.40 | 0.37 | 16.94 | 1.94 | 381 |
| HMDSO | | | | | | |

TABLE 4
Table 4: Comparison of characteristic parameters from
eq[1] (see above) for BPL-0 and BPL-1, BPL-2 and BPL-3
modified with HMDSO plasma polymer for 3 different
deposition times (1, 5, 10 min respectively) from
isopropanol adsorption at 303 K.

| | Plasma | | | | | |
|---|---|---|---|---|---|---|
| | Deposition | V | W₀ | E₀ | L | Sₘᵢ |
| Sample | Time (min) | [cm³/g] | [cm³/g] | [KJ/mol] | [nm] | [m²/g] |
| BPL | — | 0.49 | 0.45 | 17.11 | 1.89 | 480 |
| BPL | 1 | 0.45 | 0.40 | 17.11 | 1.89 | 426 |
| HMDSO | | | | | | |
| BPL | 5 | 0.40 | 0.36 | 18.74 | 1.47 | 492 |
| HMDSO | | | | | | |
| BPL | 10 | 0.41 | 0.36 | 19.09 | 1.40 | 513 |
| HMDSO | | | | | | |

TABLE 5
Table 5: Comparison of characteristic parameters from
eq[1] (see above) for BPL-0 and BPL-1, BPL-2 and BPL-3
modified with HMDSO plasma polymer for 3 different
deposition times (1, 5, 10 min respectively) from toluene
at 303 K.

| | Plasma | | | | | |
|---|---|---|---|---|---|---|
| | Deposition | V | W₀ | E₀ | L | Sₘᵢ |
| Sample | Time (min) | [cm³/g] | [cm³/g] | [KJ/mol] | [nm] | [m²/g] |
| BPL | — | 0.48 | 0.42 | 22.12 | 1.00 | 839 |
| BPL | 1 | 0.43 | 0.38 | 22.12 | 1.00 | 767 |
| HMDSO | | | | | | |
| BPL | 5 | 0.43 | 0.38 | 20.99 | 1.12 | 679 |
| HMDSO | | | | | | |
| BPL | 10 | 0.42 | 0.36 | 21.761 | 1.04 | 705 |
| HMDSO | | | | | | |

The actual values for onset of positive (Type C) curvature and negative (Type A) deviations from linearity are given in Table 6.

TABLE 6
Table 6: Points of Type A and C deviations from linearity
of DR plots.

| | Carbon | | Methanol | | | | Ethanol | |
|---|---|---|---|---|---|---|---|---|
| | Type C | | Type A | | Type C | | Type A | |
| | LnN²P⁰/P | P/P⁰ | LnN²P⁰/P | P/P⁰ | LnN²P⁰/P | P/P⁰ | LnN²P⁰/P | P/P⁰ |
| BPO-0 | 0.48 | 0.50 | 3.92 | 0.138 | 0.357 | 0.55 | 10.20 | 0.041 |
| BPL-1 | 0.19 | 0.64 | 4.71 | 0.114 | 0.357 | 0.55 | 10.20 | 0.041 |
| BPL-2 | 0.19 | 0.64 | 4.71 | 0.114 | 0.357 | 0.55 | 10.20 | 0.041 |
| BPL-3 | 0.19 | 0.64 | 4.71 | 0.114 | 0.357 | 0.55 | 10.20 | 0.041 |
| | Isopropanol | | | | Toluene | | Nitrogen | |
| | Type C | | Type A | | Type C | | Type C | |
| | LnN²P⁰/P | P/P⁰ | LnN²P⁰/P | P/P⁰ | LnN²P⁰/P | P/P⁰ | LnN²P⁰/P | P/P⁰ |
| BPO-0 | 0.40 | 0.53 | 10.20 | 0.041 | 2.94 | 0.18 | 21.49 | 0.097 |
| BPL-1 | 0.40 | 0.53 | 18.22 | 0.014 | 2.29 | 0.22 | 21.49 | 0.097 |
| BPL-2 | 0.40 | 0.53 | 18.22 | 0.014 | 2.75 | 0.19 | 21.49 | 0.097 |
| BPL-3 | 0.83 | 0.40 | 12.78 | 0.028 | 2.75 | 0.19 | 21.49 | 0.097 |

TABLE 7
Tabel 7: Enthalpy of immersion for BPL and BPL treated by
HMDSO so including the heat that comes from the ampoule.

| | Enthalpy of | Enthalpy of | Enthalpy of |
|---|---|---|---|
| | immersion | immersion | immersion |
| Plasma | _{D}H₁ (H₂O) | _{D}H₁ (Methanol) | _{D}H₁ (Isopropanol) |
| deposition | [J/g] | [J/g] | [J/g] |
| — | 40.56 | | −102.45 |
| 1 | −15.51 | −44.30 | −35.51 |
| 5 | −17.20 | −37.36 | −38.13 |
| 10 | −12.06 | −34.18 | −34.93 |

TABLE 8
Table 8: Average surface concentration for different
plasma times onto BPL, Survey scan.

| Plasma | | | |
|---|---|---|---|
| deposition | | | |
| time [min] | % O 1s | % C1s | % F 2p |
| — | 08.54 | 91.46 | — |
| 1 | 04.42 | 35.17 | 60.41 |
| 5 | — | 54.41 | 45.59 |
| 10 | | | |

The most interesting finding when all of the data sets are compared in terms of adsorbed volumes is that only for nitrogen adsorption at 77 K is the adsorption capacity in terms of W₀ significantly decreased by the surface modification process. The adsorbed volumes derived from the isotherms for the organic vapours show only very small decreases for these parameters with the greatest loss of volume occurring for the adsorbates having the highest molecular size. Inspection of the corresponding E₀ and L values show when the adsorbate is one of the alcohols tested small losses in porosity occur in the narrower supermicropore region so that adsorption occurs mainly in supermicropores toward the tope end of this size group.

This data evidences the conclusion that the major effect of the modification occurs at the entrances of the pores probably leading to constriction of their openings. This means that nitrogen at 77 K is excluded because it does not have sufficient kinetic energy to overcome the activation energy of diffusion. Clearly the majority of the pore volume is unaffected by the modification as seen from the adsorption volumes from the alcohol and toluene data.

Calorimetric measurements show that the enthalpies of immersion in water (−ΔH, H₂O) decrease from −40.56 J g⁻¹ for BPL-0 to −15.51, −17.20 and 12.06 for BPL-1, BPL-2 and BPL-3 respectively indicating that the treatment produces a degree of surface hydrophobicity.

### INDUSTRIAL APPLICABILITY OF THE INVENTION

The invention described herein offers several advantages in providing inter alia a novel microporous carbon adsorbent, in granular or fibre form, which has been treated by plasma enhanced chemical vapour deposition (PECVD) with restricted infiltration in order to modify and control only the external surface properties of the granule or fibre to control adsorption.

These new materials may be produced with hydrophobic or hydrophilic external surfaces and with variable (controllable) external pore entrances. The internal volume and surface of the porosity is not modified by the PECVD. The materials may be used in the as prepared form or after a further thermal stabilisation step which imparts refractory characteristics.

The materials are therefore size selective molecular sieve adsorbents with novel uses in a wide range of adsorption and separation applications. The hydrophobic materials have particular relevance for use in breathing apparatus and for other separation processes undertaken in high relative humidity (RH) environments.

Thus the invention finds utility in application such as, for example: hydrogen enrichment; CO₂ capture; methane storage; military & civilian respiratory protection; water scrubbing; pharmaceutical and bio separations and cigarette filters.

Various modifications and variations to the described embodiments of the invention will be apparent to those skilled in the art without departing from the scope and spirit of the invention. Although the invention has been described in connection with specific preferred embodiments, it should be understood that the invention as Claimed should not be unduly limited to such specific embodiments. Indeed, various modifications of the described modes of carrying out the invention which are obvious to those skilled in the art are intended to be covered by the present invention.

1. A microporous adsorbent particulate material having internal and external surfaces, said external surfaces
(i) having pores therein capable of admitting fluids, and
(ii) being modified by presence of a nanolayer of a polymeric material; said nanolayer extending over said external surface around a pore to partially occlude same to a predetermined extent;
said internal surfaces being substantially free of said polymeric material.

2. A microporous adsorbent material claimed in claim 1, wherein the particulate material is a carbon-based microporous material, and said internal surfaces exhibit properties associated with microporous carbon.

3. A microporous adsorbent material claimed in claim 1, wherein the polymeric material is a silicon-based material and said external surfaces exhibit properties associated with silicon.

4. A microporous adsorbent material claimed in claim 1, wherein the particulate material comprises a composite microporous material, wherein the bulk of the microporous material consists of carbon or silicon, and the surface nanolayer comprises at least one element conferring enhanced hydrophobic properties to said surface nanolayer.

5. A microporous adsorbent material claimed in claim 4, wherein the surface nanolayer comprises a halogen.

6. A microporous adsorbent material claimed in claim 5, wherein the halogen is fluorine.

7. A microporous adsorbent material claimed in claim 5, wherein the halogen is chlorine.

8. A microporous adsorbent material claimed in claim 4, wherein said nanolayer comprises a surface modifying compound to enhance hydrophobic properties of the external surfaces of the material.

9. A microporous adsorbent material claimed in claim 8, wherein the nanolayer comprises a halocarbon.

10. A microporous adsorbent material claimed in claim 8, wherein the nanolayer comprises a fluorocarbon.

11. A microporous adsorbent material claimed in claim 8, wherein the nanolayer incorporates a perfluorohexane.

12. A microporous adsorbent material claimed in claim 1, where the material is a granular particulate.

13. A microporous adsorbent material claimed in claims 1, where the particulate material is in fibre form.

14. A microporous adsorbent material claimed in claim 12, wherein the largest dimension of the particulate material has a size range of the order of nanometers to several millimeters.

15. A microporous adsorbent material claimed in claim 1, wherein the nanolayer has thickness in the range of from 1 to 1000 nanometers.

16. A microporous adsorbent material claimed in claim 1, wherein the nanolaycr comprises a hydrophobic polymer.

17. A microporous adsorbent material claimed in claim 1, wherein the nanolaycr comprises a hydrophilic polymer.

18. A microporous adsorbent material claimed in claim 1, wherein the nanolayer is a plasma enhanced chemical vapour deposit.

19. A microporous adsorbent material claimed in claim 18, wherein the nanolayer is a polymer derived from polymerisable organic precursors selected from hexadimethylsiloxane, other silanes and Si containing organics, halohydrocarbons, fluorohexane and other F-containing organics, and ChloroFluoroCarbons (CFCs/Freon type molecules).

20. A microporous adsorbent material claimed in claim 18, wherein the nanolayer is a polymer derived from oxygen-functionalised organics

21. An element comprising a microporous adsorbent material claimed in claim 1 attached to a support.

22. The element claimed in claim 21, wherein the support is selected from fibres, non-woven fibre cloths, woven fibre cloths, and flexible films.

23. The element claimed in claim 21, wherein the support comprises a fluid-permeable body.

24. The element claimed in claim 21, wherein the support comprises a carbon monolith.

25. A filter device comprising an element as claimed in claim 21.

26. A bulk storage device for fluids comprising a container filled with the microporous adsorbent material claimed in claim 1.

27. A separation system for selectively extracting one or more fluids comprising a filter device claimed in claim 25 and the bulk storage device according to claim 20, operatively connected such that selectively extracted fluid is transferable to said bulk storage device.

28. The separation system claimed in claim 27, wherein the filter device comprises microporous material adapted to selectively adsorb a gas.

29. The separation system claimed in claim 28, wherein the gas is methane.

30. The separation system claimed in claim 28, wherein the gas is carbon dioxide.

31. The separation system claimed in claim 28, wherein the gas is hydrogen.

32. A process for modifying the surface diffusion and wetting characteristics of a microporous material comprising the steps of:
converting a composition comprising a monomer to the form of a plasma;
initiating polymerisation of the composition; and
applying a nanolayer of the plasma composition to a surface of the microporous material to form a modified microporous material.

33. A process claimed in claim 32, wherein the adsorption properties of the microporous material arc modified by application of said nanolayer.

34. The process claimed in claim 32, where the hydrophobicity of the microporous material is increased.

35. The process claimed in claim 32, wherein the capacity of absorption of the microporous material is at least 90% of the capacity of adsorption of the non-modified microporous material.

36. The process claimed in claim 32, wherein the chemical and physical properties of the interior of the microporous material are not affected by application of the nanolayer.

37. The process claimed in claim 32, wherein the nanolayer is located on the surface of the microporous material and does not penetrate into the pores of the microporous material.

38. The process claimed in claim 32, wherein application of the nanolayer of the plasma composition to the microporous material takes 10 minutes or less.

39. The process claimed in claim 32, wherein application of the nanolayer of the plasma composition to the microporous material takes 1, 3 or 5 minutes.

40. The process claimed in claim 32, wherein the nanolayer of plasma composition is applied to the microporous material via a plasma enhanced chemical vapour deposition method.

41. The process claimed in claim 32, wherein a nanolayer of the plasma composition is applied to all external surfaces of the microporous material.

42. A modified microporous material obtainable by the process claimed in claim 32.

43. A modified microporous material comprising a microporous material having a nanolayer of polymer on at least one surface thereof wherein the polymer does not substantially infiltrate the pores of the microporous material, said modified microporous material allowing selective adsorption of an adsorbate dependent on the kinetic energy, molecular size and/or polarity of the adsorbate.

44. The material of claim 43 wherein the microporous material is carbon or silicon.

45. The material of claim 43, wherein the polymer is formed from hexamethyldisiloxane (HMDSO) or perfluorohexane (PFH) monomers.

46. The material of claim 43, wherein the polymer nanolaycr penetrates into the pores of the microporous material less than 0.5 nanometers.

47. The material of claim 43, wherein at least 95% of the pores on the surface of the modified microporous material arc not closed by being covered by the polymer nanolayer.

48. The material of claim 43, wherein the entrances to the pores on the surface of the modified microporous material are constricted by 50% or more relative to unmodified microporous material.

49. The material of claim 43, wherein: (i) increasing the kinetic energy of the adsorbate material increases the adsorption thereof: (ii) increasing the average molecular size of the adsorbate material decreases the adsorption thereof; and/or (iii) increasing the polarity of the adsorbate material decreases the adsorption thereof.

50. The material of claim 43, wherein the adsorbate is an alkyl molecule or an alcohol.

51. (canceled)

52. The process for selectively adsorbing a molecule comprising the step of using a modified microporous material as claimed in claim 1, for contacting a fluid containing said molecule.

53. The process claimed in claim 52, wherein the adsorption step involves the selective recovery of hydrogen from air.

54. The process according to claim 52, wherein the adsorption step involves the selective recovery of at least one of hydrogen, carbon monoxide, methane and a volatile hydrocarbon from a hydrocarbon production effluent fluid.

55. A medical apparatus comprising a gas separation element comprising a microporous material as claimed in claim 1 for selective removal of a gas or purification of a gas supply.

56. Breathing apparatus comprising microporous material as claimed in claim 1 operatively located with respect to an airway to enable selective removal of a gas or purification of a breathable gas supply.

57. The microporous adsorbent material claimed in claim 13, wherein the largest dimension of the particulate material has a size range of the order of run to several mm.

58. The process for selectively adsorbing a molecule comprising the step of using a modified microporous material as claimed in claim 43, for contacting a fluid containing said molecule.

59. The process claimed in claim 52, wherein the adsorption step involves the selective removal of toxic gases from air.

60. A medical apparatus comprising a gas separation element comprising a microporous material as claimed in claim 42 for selective removal of a gas or purification of a gas supply.

61. Breathing apparatus comprising microporous material as claimed in claim 43 operatively located with respect to an airway to enable selective removal of a gas or purification of a breathable gas supply.
